(19) United States
(12) Patent Application Publication
(10) Pub. No.: US 2012/0219647 A1
(43) Pub. Date: Aug. 30, 2012

(54) ANTIOXIDANT COMPOSITION

(76) Inventors: Harald Breivik, Porsgrunn (NO); Vidar Moen, Skien (NO); Tanja Vojnovic, Sijian (NO)

(21) Appl. No.: 13/392,632

(22) PCT Filed: Jul. 20, 2010
(86) PCT No.: PCT/NO2010/000286

§ 371 Date: May 14, 2012

(30) Foreign Application Priority Data
Aug. 26, 2009 (NO) 20092899
Mar. 17, 2010 (NO) 20100390

(51) Int. Cl. A61K 31/23; A61K 36/53; A61P 39/06; A61K 31/355

(52) U.S. Cl. 424/745; 514/547; 514/458

(57) ABSTRACT

The present invention relates to compositions comprising an edible oil having improved oxidative stability and methods for manufacturing such compositions. The composition contains an active fraction of a green tea extract, partial glycerides in an amount of at least 2% and an edible oil. The partial glycerides may contain mono- and/or diglycerides and the edible oil may be a polyunsaturated oil, e.g. a fish oil.

### FIELD OF THE INVENTION

The present invention relates to compositions comprising an edible oil having improved oxidative stability and methods for manufacturing such compositions.

### BACKGROUND OF THE INVENTION

Omega-3 fatty acids are considered essential fatty acids, which means that they are essential to human health but cannot be synthesised by the human body. For this reason, omega-3 fatty acids must be obtained through the diet.

The European Pharmacopoeia defines the omega-3 fatty acids as the following acids (see for example Monograph no. 1912, Fish Oil, Rich in Omega-3-Acids): alpha-linolenic acid (C18:3 n-3; ALA), moroctic acid (C18:4 n-3), eicosatetraenoic acid (C20:4 n-3), eicosapentaenoic (timnodonic) acid (C20:5 n-3; EPA), heneicosapentaenoic acid (C21:5 n-3), docosapentaenoic (clupanodonic) acid (C22:5 n-3) and docosahexaenoic (cervonic) acid (C22:6 n-3; EPA). Omega-3 fatty acids with chain-length of 20 and above are called long-chain omega-3 fatty acids. ALA is common in a number of vegetable oils. C18:4 n-3 is available from fish oils, as well as from some vegetable oils. Once eaten, the body can to some extent convert ALA and C18:4 n-3 to the long-chain omega-3 fatty acids, including EPA and DHA. However, fish oil and other marine oils are known to be the best source of these omega-3 fatty acids. Long-chain omega-3 fatty acids can also be obtained via fermentation of single cell oils (microbial oils), and research projects aim at producing EPA and DHA via gene-modified terrestrial plants.

Omega-3 fatty acids have been demonstrated to reduce the risk of coronary heart disease as well as having a positive effect on children's development, as well as on the skin. Results have also been disclosed indicating the positive effect of these fatty acids on certain mental illnesses, autoimmune diseases and joint complaints. There are therefore many reasons for considering taking fish oil as a valuable dietary supplement, including the long-term effect which this dietary supplement is now thought to have.

However, fish oils and especially concentrates of omega-3 fatty acids are very, susceptible to oxidation. Oxidation limits the use of such products in food applications, and also limits oral administration of omega-3 containing nutritional supplements, except where the supplements are encapsulated.

Microencapsulation is a way of formulating omega-3 oils for food applications. However, microencapsulated products are relatively expensive, the encapsulation material often takes up more volume than the oil, so that the total volume becomes impractical to handle, and also there might be doubts whether the encapsulation material prevents the valuable omega-3 fatty acids from being absorbed in the intestinal tract.

An alternative to microencapsulation is the use of antioxidants in the oil to slow down oxidation. Most antioxidants interfere with the propagation of lipid oxidation by donating a hydrogen atom to, and thereby inactivating, chain-carrying peroxyl, radicals and/or alkoxyl radicals. Hence, after breaking the chain reaction of lipid peroxidation, an antioxidant is itself converted to a radical. To be effective, the antioxidant radical has to be sufficiently stable so as to react slowly with the lipid substrate and rapidly with lipid peroxyl radicals and/or alkoxyl radicals. Several natural and synthetic compounds fulfil this condition and are widely used for preserving polyunsaturated fatty acids (PUFA) from oxidative deterioration. Even though a number of synthetic antioxidants have been extensively used for the stabilization of foods, much interest has developed in the use of naturally occurring antioxidants because of the adverse attention received by the synthetic antioxidants and because of the worldwide trend to avoid or minimize the use of synthetic food additives.

Tocopherols are among the most important lipid-soluble natural antioxidants, and appear to be the major physiological scavengers of free radicals inside human membranes and plasma lipids. The fact that these compounds are naturally occurring lipid-soluble antioxidants make them particularly useful in combination with marine oils, having high amounts of PUFA, intended for human consumption (Free radical biology & medicine, 2005, vol. 38, page 78-84; J. Chem. Soc., Perkin Trans. 2, 1998).

Another example of antioxidants commonly used in combination with marine oils are rosemary extracts. The antioxidant potential of such an extract has previously been tested on cod liver oil and has been shown to have significantly higher antioxidant effect compared with seven other naturally occurring antioxidants (Journal of Aquatic Food Product Technology; vol. 14; 2005; page 75-94). Further, a mixture of α-tocopherol and rosemary extract has previously been shown to exert very strong antioxidant activity in sardine oil, where their combination not only inhibited the formation of hydroperoxides much more effectively than when present separately but the activity of tocopherol was retained for a longer period of time (Yukagaku 1994, vol. 43, no 2, page 109-115).

Ascorbyl palmitate is an ester formed from ascorbic acid and palmitic acid. In addition to its use as a source of vitamin C, it is also commonly used as an antioxidant food additive. The compound is difficult to dissolve in oil formulations, and it is therefore common to add lecithin to the antioxidant preparation in order to solubilise the ascorbyl palmitate (EP612346). Even though lecithin is commonly regarded as a well-tolerated and non-toxic surfactant, lecithin may contain traces of proteins and for this reason has to be declared as a potential allergen in nutritional supplements. Producers and distributors prefer to avoid components that might act as allergens.

In addition to traditional and cultural reasons for consuming tea, a renewed interest has been fuelled by the discovery of strong antioxidant properties provided by tea prepared from Camellia Sinensis leaves (green tea). Such an antioxidant effect has primarily been attributed to the polyphenol content of the tea leaves, commonly known as tea catechins. Said catechins are water-soluble and therefore not easily dissolvable in oil formulations. In order to make these strong antioxidants lipid-soluble, it has been suggested to derivatise part of the phenols with fatty acid (WO07021789). An unwanted side effect of derivatising these compounds with fatty acids is that the intestinal absorption of these compounds increases significantly. Polyphenols from green tea are generally not absorbed into the body, and the increased absorption of these compounds add a problem from a regulatory point of view.

Even though a number of antioxidants and various combinations thereof have been disclosed (e.g. U.S. Pat. No. 5,102,659), there is still a need for additional antioxidant compositions having improved characteristics.

### SUMMARY OF THE INVENTION

It has previously been disclosed that green tea extracts have strong antioxidant activity. However, most of the compounds responsible for said antioxidant activity are water-soluble and therefore not easily dissolvable in oil formulations. It has been suggested to derivatise the active compounds with fatty acids, but since this may add a problem from a regulatory point of view, it is desirable to avoid this strategy.

Accordingly, it is an object of the present invention to provide a composition comprising an edible oil, preferably an edible marine oil, and green tea extract without having to chemically modify the green tea catechines.

Surprisingly, the inventors of the present invention have observed that the solubility of the green tea catechines is highly dependent on the content of partial glycerides in the is composition.

Thus, a first aspect of the present invention relates to a composition comprising an edible oil and green tea extract or an active fraction of said green tea extract, said composition containing more than 2% partial glycerides by weight of the composition.

As previously disclosed, partial glycerides have been shown to significantly increase the solubility of green tea catechines in oily formulations. Of particular interest are citric acid and tartaric acid esters of partial glycerides which have been shown especially suitable for dissolving green tea catechines in oily formulations.

Thus, a third aspect of the present invention relates to composition comprising an edible oil, green tea extract or an active fraction of said green tea extract and component A, wherein component A is a compound of formula (I) or a salt thereof; or a mixture of different compounds each represented by formula (I) or a salt thereof,

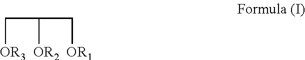

wherein
R₁ represents

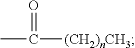

n is an integer in the range 3-30;
R₂ and R₃ are independently selected from the group consisting of

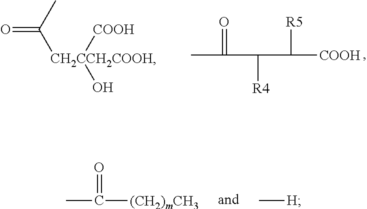

m is an integer in the range 3-30;
R₄ and R₅ are independently selected from

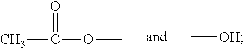

with the proviso that at least one of R₂ and R₃ is other than

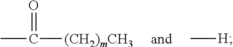

and that at least one of R₄ and R₅ is —OH.

A second aspect of the present invention relates to a method for manufacturing the composition according to present invention, the method comprising the following steps:
a) optionally, green tea extract or an active fraction thereof is mixed with ascorbyl palmitate;
b) an edible oil is mixed with partial glycerides so that the final composition contains more than 2% partial glycerides by weight of the final composition (if said edible oil contains more than 2% partial glycerides by weight of the final composition, it is not necessary to add further partial glycerides); or an edible oil is mixed with component A, as defined in the third aspect of the present invention;
c) green tea extract or an active fraction thereof or the product obtained in step a) is mixed with the product obtained in step b);
d) the product obtained in step c) is subjected to filtration;
e) optionally, at least one tocopherol or tocopherol derivative and/or rosemary extract or an active fraction thereof is added to the product obtained in step a), b), c) and/or d).

A fourth aspect of the present invention relates to a method for manufacturing the composition according to the present invention, the method comprising the following steps:
a) green tea extract or an active fraction thereof is mixed with a lower alcohol (C₁₋₆ alcohol), such as ethanol;
b) the product obtained in step a) is subjected to filtration;
c) optionally, the filtrate obtained in step b) is mixed with ascorbyl palmitate;
d) component A, as defined in the third aspect of the present invention, or partial glycerides, as defined in the first aspect of the present invention is added to the product obtained in step b) or step c); or optionally directly to the edible oil defined in step g);
e) optionally, at least one tocopherol or tocopherol derivative and/or rosemary extract or an active fraction thereof is added to the product obtained in step a), b), c) or d);
f) the lower alcohol is removed; and
g) the product obtained in step f) is mixed with an edible oil.

A fifth aspect of the present invention relates to a nutritional, food or pharmaceutical composition, comprising the composition according to the first or third aspect of the present invention or the composition obtainable by the method according to the second or fourth aspect of the present invention.

### DESCRIPTION OF THE FIGURES

FIG. 1 shows a synergistic antioxidative effect (induction time analysis) from combining a mixture of tocopherols (Tocoblend L 70 IP, Vitablend (1.03 mg per gram marine oil)), rosemary extract (Oleoresin Rosemary, Kalsec (2.87 mg per gram marine oil)) and ascorbyl palmitate (Grindox ascorbyl palmitate, Danisco (0.72 mg per gram marine oil)) and olive oil (0.78 mg olive oil per gram marine oil) with green tea extract (GUARDIAN™ Green Tea Extract 20M, Danisco (5.4 mg per gram marine oil before filtration).

1 represents Ascorbyl Palmitate+Tocopherols+Rosemary extract;

2 represents Green Tea Extract;

3 represents 1+2, “filtration, 20° C.”;

4 represents 1+2, “filtration, 80° C.”

FIG. 2 shows a synergistic antioxidative effect (increased weight analysis) from, combining a mixture of tocopherols (Tocoblend L 70 IP, Vitablend (1.03 mg per gram marine oil)), rosemary extract (Oleoresin Rosemary, Kalsec (2.87 mg per gram marine oil)) and ascorbyl palmitate (Grindox ascorbyl palmitate, Danisco (0.72 mg per gram marine oil)) and olive oil (0.78 mg olive oil per gram marine oil) with green tea extract (GUARDIAN™ Green Tea Extract 20M, Danisco (5.4 mg per gram marine oil before filtration).

1 represents 2+ Green Tea Extract, “filtration, 80° C.”;

2 represents 4+ Ascorbyl Palmitate+Tocopherols+Rosemary extract;

3 represents 2′-Green Tea Extract, “filtration, 20° C.”;

4 represents EPAX 6000 EE;

FIG. 3 shows the amount of green tea catechines dissolved in water and four different oil preparations. Dissolution in water gives a straight line that can be utilised as a calibration curve for estimation of the dissolved amount in the omega-3 containing oils.

| | |
|---|---|
| Acid | number that expresses in milligrams the additional quantity of |
| value | potassium hydroxide compared to the composition without green |
| | tea extract required to neutralise 1 gram of the composition. |
| Mg/g | amount of catechins per gram oil |
| active | |
| Water | Water + guardian 20M (green tea extract) |
| EE1 | marine oil (5.6% triglycerides (TG); 3.0% diglycerides (DG); |
| | 3.6% monoglycerides (MG); 87.8% Ethyl esters/Free fatty acids |
| | (EE/FFA)) + guardian 20M (green tea extract) |
| EE2 | marine oil (0% triglycerides (TG); 0.5% diglycerides (DG); |
| | 12.0% monoglycerides (MG); 87.5% Ethyl esters/Free fatty |
| | acids (EE/FFA)) + guardian 20M (green tea extract) |
| TG1 | marine oil (93.6% triglycerides (TG); 4.9% diglycerides (DG); |
| | 0.4% monoglycerides (MG); 0.5% Ethyl esters/Free fatty acids |
| | (EE/FFA)) + guardian 20M (green tea extract) |
| TG2 | marine oil (63.9% triglycerides (TG); 31.6% diglycerides (DG); |
| | 1.9% monoglycerides (MG); 2.5% Ethyl esters/Free fatty acids |
| | (EE/FFA)) + guardian 20M (green tea extract) |

FIG. 4 shows the amount of green tea catechines dissolved in water and a marine oil mainly containing ethyl esters and free fatty acids) with or without citrem. Dissolution in water gives a straight line that can be utilised as a calibration curve for estimation of the dissolved amount in the marine oil.

| | |
|---|---|
| Acid value | number that expresses in milligrams the additional quantity |
| | of potassium hydroxide compared to the composition without |
| | green tea extract required to neutralise 1 gram of the |
| | composition. |
| Mg/g | amount of catechins per gram oil |
| active | |
| Water | Water + guardian 20M (green tea extract) |

FIG. 5 shows the amount of green tea catechines dissolved in a marine oil (mainly containing triglycerides) with or without citrem.

| | |
|---|---|
| Acid | number that expresses in milligrams the additional quantity |
| value | of potassium hydroxide compared to the composition without |
| | green tea extract required to neutralise 1 gram of the composition. |
| Mg/g | amount of catechins per gram oil |
| active | |

FIG. 6 shows the amount of green tea catechines dissolved in a marine oil (mainly containing ethyl esters and free fatty acids) with or without 2.1% monoglycerides by weight of the final composition.

| | |
|---|---|
| Acid | number that expresses in milligrams the additional quantity |
| value | of potassium hydroxide compared to the composition without |
| | green tea extract required to neutralise 1 gram of the composition. |
| Mg/g | amount of catechins per gram oil |
| active | |

### DETAILED DESCRIPTION OF THE INVENTION

As previously disclosed, there are many reasons for considering taking fish oil as a valuable dietary supplement, including the long-term effect which this dietary supplement is now thought to have. However, fish oils and especially concentrates of omega-3 fatty acids are very susceptible to oxidation. Accordingly, there is a need in the art to provide an edible oil composition, particularly an edible marine oil composition, having improved oxidative stability.

It has previously been disclosed that green tea extracts contain compounds which have strong antioxidant activity. However, most of the compounds responsible for said antioxidant activity are water-soluble and therefore not easily dissolvable in oil formulations. It has been suggested to derivatise the active compounds with fatty acids (WO07021789), but since this may add a problem from a regulatory point of view, it is desirable to avoid this strategy.

Accordingly, it is an object of the present invention to provide a composition comprising an edible oil, preferably an edible marine oil, and green tea extract without having to chemically modify the green tea catechines.

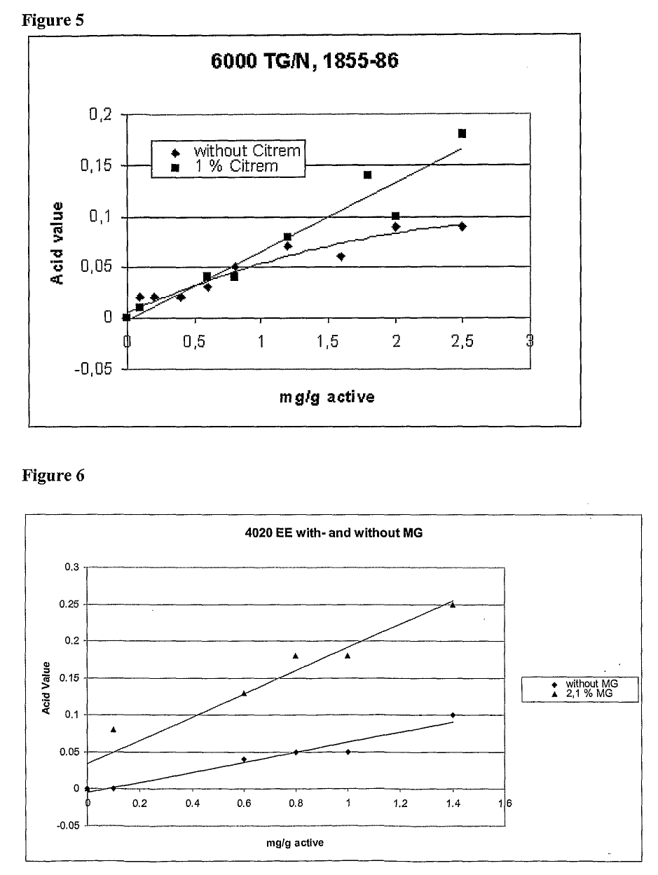

The antioxidant effect of green tea has primarily been attributed to the polyphenol content of the tea leaves, commonly known as tea catechines. Based on the assumption that the active components of green tea extract to a large extent are present as compounds like phenols, the dissolved amount may be analysed by titration with potassium hydroxide. This approach has been used in order to investigate a) whether the amount of monoglycerides, diglycerides and triglycerides in the composition may affect dissolution of the green tea components (example 7, FIG. 3, FIG. 6); and b) whether there are any specific compounds that are particularly useful when it comes to increasing the amount of green tea components that may be dissolved in oil formulations (example 8, example 10, FIG. 4 and FIG. 5).

The results of the experiments disclosed in example 7 clearly indicate that when green tea extract on maltodextrin carrier is added according to the present invention to an is ethyl ester or triglyceride oil containing partial glycerides, the green tea component that can be analysed by titration with potassium hydroxide can be dissolved nearly quantitatively in the oil. For a triglyceride with very low amounts of partial glycerides the dissolution is much lower. This positive dissolution promoting effect of the partial glycerides is very surprising.

Further, the results of the experiments disclosed in example 11 clearly indicate that when green tea extract on maltodextrin carrier is mixed with an ethyl ester to which monoglycerides have been added, the amount of green tea components that can be analysed by titration with potassium hydroxide is significantly higher as compared to control (ethyl ester without added monoglycerides).

Thus, a first aspect of the present invention relates to a composition comprising an edible oil and green tea extract or an active fraction of said green tea extract, said composition containing more than 2% partial glycerides by weight of the composition.

In one embodiment according to the present invention, the composition contains more than 2.5% partial glycerides by weight of the composition, preferably at least 3% partial glycerides by weight of the composition (e.g. at least 4% by weight of the composition), more preferably at least 5% partial glycerides by weight of the composition (e.g. at least 6% or 7% by weight of the composition), even more preferably at least 8% partial glycerides by weight of the composition (e.g. at least 9% by weight of the composition) and most preferably at least 10% partial glycerides by weight of the composition (e.g. at least 15%, 17%, 19%, 21%, 23% or 25% by weight of the composition).

Partial glycerides are esters of glycerol with fatty acids, where not all the hydroxyl groups are esterified (mono- and/or diglycerides).

In one embodiment according to the present invention, said partial glycerides are monoglycerides.

Accordingly, the present invention relates to a composition comprising an edible oil and green tea extract or an active fraction of said green tea extract, said composition containing more than 2% monoglycerides by weight of the composition.

In one embodiment according to the present invention, the composition contains more is than 2.5% monoglycerides by weight of the composition, preferably at least 3% monoglycerides by weight of the composition (e.g. at least 4% by weight of the composition), more preferably at least 5% monoglycerides by weight of the composition (e.g. at least 6% or 7% by weight of the composition), even more preferably at least 8% monoglycerides by weight of the composition (e.g. at least 9% by weight of the composition) and most preferably at least 10% monoglycerides by weight of the composition (e.g. at least 15%, 17%, 19%, 21%, 23% or 25% by weight of the composition).

In another embodiment, said partial glycerides are diglycerides.

Accordingly, the present invention relates to a composition comprising an edible oil and green tea extract or an active fraction of said green tea extract, said composition containing more than 2% diglycerides by weight of the composition.

In one embodiment according to the present invention, the composition contains more than 2.5% diglycerides by weight of the composition, preferably at least 3% diglycerides by weight of the composition (e.g. at least 4% by weight of the composition), more preferably at least 5% diglycerides by weight of the composition (e.g. at least 6% or 7% by weight of the composition), even more preferably at least 8% diglycerides by weight of the composition (e.g. at least 9% by weight of the composition) and most preferably at least 10% diglycerides by weight of the composition (e.g. at least 15%, 17%, 19%, 21%, 23% or 25% by weight of the composition).

In yet another embodiment, said partial glycerides are a mixture of mono- and diglycerides.

Accordingly, the present invention relates to a composition comprising an edible oil and green tea extract or an active fraction of said green tea extract, said composition containing more than 2% of a mixture of mono- and diglycerides by weight of the composition.

In one embodiment according to the present invention, the composition contains more than 2.5% of a mixture of mono- and diglycerides by weight of the composition, preferably at least 3% of a mixture of mono- and diglycerides by weight of the composition (e.g. at least 4% by weight of the composition), more preferably at least 5% of a mixture of mono- and diglycerides by weight of the composition (e.g. at least 6% or 7% by weight of the composition), even more preferably at least 8% of a mixture of mono- and diglycerides by weight of the composition (e.g. at least 9% by weight of the composition) and most preferably at least 10% of a mixture of mono- and diglycerides by weight of the composition (e.g. at least 15%, 17%, 19%, 21%, 23% or 25% by weight of the composition).

Said partial glycerides are preferably glycerol mono- and/or diesters of unsaturated fatty acids, preferably glycerol mono- and/or diesters of polyunsaturated fatty acids and most preferably glycerol mono- and/or diesters of omega-3 fatty acids. Said fatty acids preferably having 3-30 carbon atoms, more preferably 5-30 carbon atoms and most preferably 10-30 carbon atoms.

Polyunsaturated fatty acids are fatty acids having 2 or more double bonds, preferably separated from each other by a single methylene group. Preferably, said polyunsaturated fatty acids have more than 2 double bonds, e.g. 3, 4, 5 or 6 double bonds that preferably are separated from each other by a single methylene group.

Omega-3 fatty acids are a family of unsaturated fatty acids that have in common a final carbon-carbon double bond in the n-3 position; that is, the third bond from the methyl end of the fatty acid. Nutritionally important n-3 fatty acids include α-linolenic acid (ALA), eicosapentaenoic acid (EPA), and docosahexaenoic acid (DHA), all of which are polyunsaturated.

By the expression partial glycerides by weight of the composition or by weight of the edible oil, there is meant the results from analysis by size-exclusion chromatography according to methods similar to those described in Ph. Eur. monographs 1352 (Omega-3-acid triglycerides) and 2063 (Omega-3-acid ethyl esters 60).

By the expression omega-3 fatty acids by weight of the composition or by weight of the edible oil, there is meant not only the free omega-3 fatty acids but also omega-3 fatty acids in the form of e.g. glycerides (mono-, di- and triglycerides) and/or ethyl esters. For quantitative determination of the EPA and DHA content in omega-3 containing products and calculation of percentage content of the total omega-3 acids, see Ph. Eur. 2.4.29.

Further, the results of the experiments presented in example 8 and 10 clearly indicate that when green tea extract on maltodextrin carrier is added to an ethyl ester or triglyceride oil to which citric acid esters of partial glycerides have been added, the amount of green tea components present in the oil that can be analysed by titration with potassium hydroxide is significantly increased. This positive dissolution promoting effect of the citric acid esters of partial glycerides is very surprising.

Thus, a third aspect of the present invention relates to a composition comprising an edible oil, green tea extract or an active fraction of said green tea extract and component A, wherein component A is a compound of formula (I) or a salt thereof; or a mixture of different compounds each represented by formula (I) or a salt thereof,

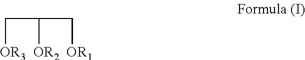

wherein
R₁ represents

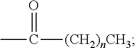

n is an integer in the range 3-30;
R₂ and R₃ are independently selected from the group consisting of

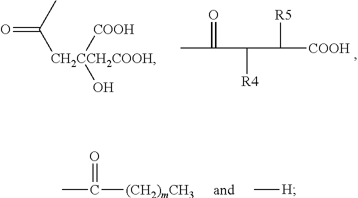

m is an integer in the range 3-30;
R₄ and R₅ are independently selected from

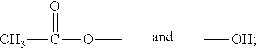

with the proviso that at least one of R₂ and R₃ is other than

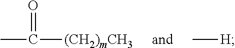

and that at least one of R₄ and R₅ is —OH.

As component A contains a plurality of acid groups, component A may exist in i) non-neutralized form; ii) partly neutralized form; iii) essentially neutralized form or iv) is completely neutralized form, mainly depending on the pH of the solution in which it is dissolved.

In one embodiment according to the present invention, component A is in non-neutralized form, more preferably in partly neutralized form and most preferably in essentially or completely neutralized form. Preferably said component A is partly, essentially or completely neutralized using sodium hydroxide or potassium hydroxide.

The compound defined as

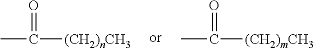

may be linear or branched, preferably linear.

It is preferred that n is an integer in the range 5-30, such as 7-30, 9-30, 11-30, 13-30, 15-30 or 16-30; more preferably n is an integer in the range 5-25, such as 7-25, 9-25, 11-25, 13-25, 15-25 or 16-25; even more preferably n is an integer in the range 5-20, such as 7-20, 9-20, 11-20, 13-20, 15-20 or 16-20; and most preferably n is an integer in the range 5-16, such as 7-16, 9-16, 11-16, 13-16, 15-16, such as e.g. 16.

Further, it is preferred that m is an integer in the range 5-30, such as 7-30, 9-30, 11-30, 13-30, 15-30 or 16-30; more preferably m is an integer in the range 5-25, such as 7-25, 9-25, 11-25, 13-25, 15-25 or 16-25; even more preferably m is an integer in the range 5-20, such as 7-20, 9-20, 11-20, 13-20, 15-20 or 16-20; and most preferably m is an integer in the range 5-16, such as 7-16, 9-16, 11-16, 13-16, 15-16, such as e.g. 16.

In one preferred embodiment m is equal to n.

In one preferred embodiment, R₂ and R₃ is independently selected from the group consisting of:

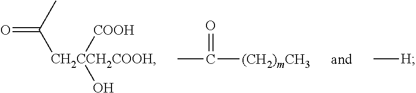

with the proviso that at least one of R₂ and R₃ is other than

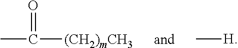

In another embodiment, R₂ and R₃ is independently selected from the group consisting of:

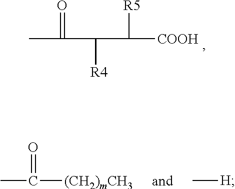

wherein R₄ and R₅ are independently selected from

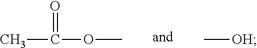

with the proviso that at least one of R₂ and R₃ is other than

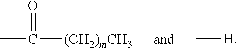

In one preferred embodiment, R₂ is —H or

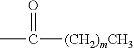

and R₃ is selected from the group consisting of:

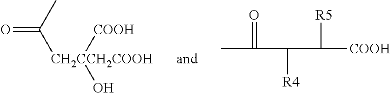

wherein R₄ and R₅ are independently selected from

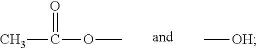

In one preferred embodiment, R₂ is —H or

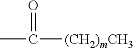

### and R₃ is

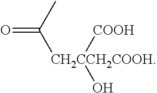

In one preferred embodiment, R₂ is —H or

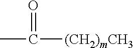

### and R₃ is

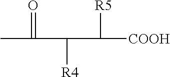

wherein R₄ and R₅ are independently selected from

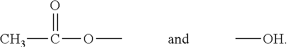

In one embodiment according to the present invention, component A is a mixture of different compounds each represented by formula (I) or a salt thereof.

In one embodiment according to the present invention, component A represents a compound of formula (II) or a salt thereof; a compound of formula (III) or a salt thereof; or any mixture thereof

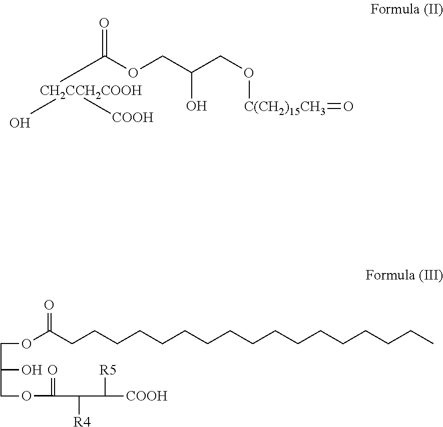

wherein R₄ and R₅ are independently selected from

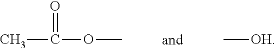

In another embodiment, both R₄ and R₅ are —OH. In another embodiment R₄ is ═OH and

### R₅ is

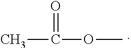

In another embodiment, R₄ is

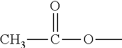

### and R₅ is —OH.

In one embodiment according to the present invention, component A represents a compound of formula (II) or a salt thereof. In another embodiment, component A represents a compound of formula (III) or a salt thereof. In another embodiment, component A represents a mixture of compounds each represented by formula II or salts thereof; or formula III or salts thereof.

In one embodiment according to the present invention the composition contains at least 0.3% component A by weight of the composition, preferably at least 0.35% component A by weight of the composition, more preferably at least 0.4% component A by weight of the composition, even more preferably at least 0.5% component A by weight of the composition and most preferably at least 0.55% component A by weight of the composition such as 0.6%, 0.65%, 0.7%, 0.75%, 0.8%, 0.85%, 0.9%, 0.95%, 1%, 1.5%, 2%, 2.5%, 3%, 3.5%, 4%, 4.5%, 5%, 5.5% or 6% by weight of the composition.

In one embodiment according to the present invention, said composition does not contain a phenolic diterpene.

Diterpenes are natural substances having 20 carbon atoms which are made up of four isoprene units. They belong to the terpene group. Compounds designated diterpenes are not only hydrocarbons, but also derivatives of the corresponding hydrocarbons. Phenolic diterpenes are those diterpenes which contain at least one phenolic OH group, e.g. Carnosolic acid.

In another embodiment according to the present invention, the only source of is triglycerides in said composition (particularly triglycerides of which each fatty acid have 6-18 carbon atoms, such as e.g. triglycerides the fatty acids of which have predominantly 8 or 10 carbon atoms) being the triglycerides that may be present in the edible oil.

In another embodiment according to the present invention, said composition does not contain triglycerides, particularly triglycerides of which each fatty acid have 6-18 carbon atoms, such as e.g. triglycerides of which each fatty acid have predominantly 8 or 10 carbon atoms. One example of a commercially available triglyceride containing composition, of which each fatty acid has predominantly 8 or 10 carbon atoms, is Delios® V.

In one embodiment according to the present invention, said composition contains less than 3% of a hydrophobic emulsifier by weight of the composition, e.g. less than 2.5% of a hydrophobic emulsifier by weight of the composition, less than 2% of a hydrophobic emulsifier by weight of the composition, less than 1.5% of a hydrophobic emulsifier by weight of the composition, less than 1% of a hydrophobic emulsifier by weight of the composition or less than 0.5% of a hydrophobic emulsifier by weight of the composition.

In another embodiment according to the present invention, a hydrophobic emulsifier is not added to the composition.

A hydrophobic emulsifier has an HLB value of less than 8. For determination of HLB values see: Tensid-Taschenbuch [surfactant Handbook], 2^{nd} edition, edited by H. Stache, Carl Hanser Verlag, 1981.

In one embodiment, said hydrophobic emulsifier is selected from the group consisting of a polyglycerol polyricinolate, a polyglycerol ester of fatty acid and glycerol monoester of fatty acid (e.g. glycerol monooleate and glycerol monostearate). In another embodiment said hydrophobic emulsifier is mono- and/or diesters of mono-unsaturated fatty acids and/or mono- and/or diesters of unsaturated fatty acids.

In one embodiment according to the present invention, said composition contains less than 3% water by weight of the composition, e.g. less than 2.5% water by weight of the composition, less than 2% water by weight of the composition, less than 1.5% water by weight of the composition, less than 1% water by weight of the composition, less than 0.5% water by weight of the composition, less than 0.05% water by weight of the composition, less than 0.01% water by weight of the composition or less than 0.005% water by weight of the composition.

In another embodiment according to the present invention, the composition is essentially or totally free of water.

By the expression “edible oil” there is meant an edible oil or an oil derived therefrom. Said edible oil may be of animal or vegetable origin. Examples of oil derived from an edible oil are ethyl esters and free fatty acids.

In one embodiment the edible oil is in the form of triglycerides, diglycerides, monoglycerdies, ethyl esters, free fatty acids or any combination thereof.

Said partial glycerides are preferably glycerol mono- and/or diesters of unsaturated fatty acids, preferably glycerol mono- and/or diesters of polyunsaturated fatty acids and most preferably glycerol mono- and/or diesters of omega-3 fatty acids. Said fatty acids preferably having 3-30 carbon atoms, more preferably 5-30 carbon atoms and most preferably 10-30 carbon atoms.

In one preferred embodiment, said edible oil is in the form of ethyl esters, preferably ethyl esters of unsaturated fatty acids (e.g. of long-chain unsaturated fatty acids) more preferably ethyl esters of polyunsaturated fatty acids (e.g. of long-chain polyunsaturated fatty acids) most preferably ethyl esters of omega-3 fatty acids (e.g. of long-chain omega-3 fatty acids).

In another preferred embodiment, said edible oil is in the form of triglycerides, diglycerides or monoglycerides or any mixture thereof, preferably of unsaturated fatty acids (e.g. of long-chain unsaturated fatty acids) more preferably of polyunsaturated fatty acids (e.g. of long-chain polyunsaturated fatty acids) most preferably of omega-3 fatty acids (e.g. of long-chain omega-3 fatty acids).

In one preferred embodiment, said edible oil is in the form of free fatty acids (e.g. long-chain free fatty acids), preferably free unsaturated fatty acids (e.g. long-chain unsaturated fatty acids), more preferably free polyunsaturated fatty acids most preferably free omega-3 fatty acids (e.g. of long-chain omega-3 fatty acids).

In one embodiment, the edible oil or an oil derived therefrom contains >40% ethyl esters by weight of the edible oil, >50% ethyl esters by weight of the edible oil, >60% ethyl esters by weight of the edible oil, preferably >70% ethyl esters by weight of the edible oil, even more preferably >80% ethyl esters by weight of the edible (Aland most preferably >90% ethyl esters by weight of the edible oil, such as e.g. >99% or 100% ethyl esters by weight of the edible oil. Said ethyl esters preferably being ethyl esters of zo omega-3 fatty acids.

In another embodiment according to the present invention, the edible oil or an oil derived therefrom contains >30% triglycerides by weight of the edible oil, preferably >35% triglycerides by weight of the edible oil, even more preferably >40% triglycerides by weight of the edible oil (e.g. >45%, >50% or >55%) and most preferably >60% triglycerides by weight of the edible oil, such as e.g. >65%, >70%, >75%, >80%, >85%, >90% or >95% triglycerides by weight of the edible oil. Said triglycerides preferably being triglycerides of omega-3 fatty acids.

In another embodiment according to the present invention, the edible oil or an oil derived therefrom contains >30% unsaturated fatty acids by weight of the edible oil, preferably >35% unsaturated fatty acids by weight of the edible oil, even more preferably >40% unsaturated fatty acids by weight of the edible oil (e.g. >45 >50% or >55%) and most preferably >60% unsaturated fatty acids by weight of the edible oil, such as e.g. >65%, >70%, >75%, >80%, >85%, >90% or >95% unsaturated fatty acids by weight of the edible oil.

In another embodiment according to the present invention, the edible oil or an oil derived therefrom contains >30% polyunsaturated fatty acids by weight of the edible oil, preferably >35% polyunsaturated fatty acids by weight of the edible oil, even more preferably >40% polyunsaturated fatty acids by weight of the edible oil (e.g. >45%, >50% or >55%) and most preferably >60% polyunsaturated fatty acids by weight of the edible oil, such as e.g. >65%, >70%, >75%, >80%, >85%, >90% or >95% polyunsaturated fatty acids by weight of the edible oil.

In another embodiment according to the present invention, the edible oil or an oil derived therefrom contains >30% omega-3 fatty acids by weight of the edible oil, preferably >35% omega-3 fatty acids by weight of the edible oil, even more preferably >40% omega-3 fatty acids by weight of the edible oil (e.g. >45%, >50% or >55%) and most preferably >60% omega-3 fatty acids by weight of the edible oil, such as e.g. >65%, >70%, >75%, >80%, >85%, >90% or >95% omega-3 fatty acids by weight of the edible oil.

In another embodiment according to the present invention, the edible oil or an oil derived therefrom contains more than 2% partial glycerides by weight of the edible oil, more preferably at least 3% partial glycerides by weight of the edible oil, even more zo preferably at least 5% partial glycerides by weight of the edible oil and most preferably at least 7% partial glycerides by weight of the edible oil, such as e.g. at least 8, 10, 12, 15, 17, 19, 21, 23, 25, 30, 35, 40, 45, 50, 55, 60, 65 or 70% partial glycerides by weight of the edible oil.

In another embodiment according to the present invention, the edible oil or an oil derived therefrom contains more than 2% diglycerides by weight of the edible oil, more preferably at least 3% diglycerides by weight of the edible oil, even more preferably at least 5% diglycerides by weight of the edible oil and most preferably at least 7% diglycerides by weight of the edible oil, such as e.g. at least 8, 10, 12, 15, 17, 19, 21, 23, 25, 30, 35, 40, 45, 50, 55, 60, 65 or 70% diglycerides by weight of the edible oil.

In another embodiment according to the present invention, the edible oil or an oil derived therefrom contains more than 2% monoglycerides by weight of the edible oil, more preferably at least 3% monoglycerides by weight of the edible oil, even more preferably at least 5% monoglycerides by weight of the edible oil and most preferably at least 7% monoglycerides by weight of the edible oil, such as e.g. at least 8, 10, 12, 15, 17, 19, 21, 23, 25, 30, 35, 40, 45, 50, 55, 60, 65 or 70% monoglycerides by weight of the edible oil.

In one embodiment according to the present invention, said edible oil or an oil derived therefrom contains less than 70% partial glycerides by weight of the edible oil, more preferably less than 60% partial glycerides by weight of the edible oil, even more preferably less than 50% partial glycerides by weight of the edible oil and most preferably less than 40% partial glycerides by weight of the edible oil, such as e.g. less than 30, 20, 10, 8, 6, 4 or 3% partial glycerides by weight of the edible oil (e.g. 2% or to less than 2% partial glycerides by weight of the edible oil).

In one embodiment according to the present invention, said edible oil or an oil derived therefrom contains less than 70% diglycerides by weight of the edible oil, more preferably less than 60% diglycerides by weight of the edible oil, even more preferably is less than 50% diglycerides by weight of the edible oil and most preferably less than 40% diglycerides by weight of the edible oil, such as e.g. less than 30, 20, 10, 8, 6, 4 or 3% diglycerides by weight of the edible oil (e.g. 2% or less than 2% diglycerides by weight of the edible oil).

In one embodiment according to the present invention, said edible oil or an oil derived therefrom contains less than 70% monoglycerides by weight of the edible oil, more preferably less than 60% monoglycerides by weight of the edible oil, even more preferably less than 50% monoglycerides by weight of the edible oil and most preferably less than 40% monoglycerides by weight of the edible oil, such as e.g. less than 30, 20, 10, 8, 6, 4 or 3% monoglycerides by weight of the edible oil (e.g. 2% or less than 2% monoglycerides by weight of the edible oil).

By the expression “marine oil” there is meant a marine oil or an oil derived therefrom. Examples of oils derived from an edible oil are ethyl esters, free fatty acids and omega-3 fatty acid concentrates. Preferably, said marine oil is an edible marine oil, such as fish oil; and an oil derived therefrom is preferably ethyl esters (preferably ethyl esters of omega-3 fatty acids such as e.g. ethyl ester concentrates of omega-3 fatty acids), free fatty acids or omega-3 fatty acid concentrates produced from a marine oil such as e.g. fish oil.

The composition according to the present invention comprises green tea extract or an active fraction thereof. By the expression “green tea extract or an active fraction thereof” there is meant any extracts obtainable from green tea which have antioxidant activity. Antioxidant activity might be measured by a number of methods such as the Oxipres method disclosed in example 2.

The major tea catechins are epigallocatechin gallate (EGCG), epigallocatechin (EGC), epicatechin gallate (ECG), and epicatechin (EC). Of these, EGCG is the most abundant and possesses the most potent antioxidative activity.

Thus, it is preferred that said green tea extract, or an active fraction thereof, comprises at least one catechin, more preferably the green tea extract comprises epigallocatechin gallate (EGCG), epigallocatechin (EGC), epicatechin gallate (ECG) or epicatechin (EC) or any mixture thereof, and even more preferably it comprises epigallocatechin gallate (EGCG).

It is preferred that the total amount of catechin(s) in said green tea extract, or an active fraction thereof, is as high as possible, e.g. in the range 1-80%, in the range 10-70%, in the range 20-50% or in the range 20-30% by weight of the extract.

Further, it is preferred that said green tea extract is added to a carrier. As shown in example 3, a composition prepared using green tea extract on a carrier was found significantly more stable than a composition prepared using green tea extract without a carrier. Further, the carrier alone was found not to affect the stability of the composition. These results indicate that more of the active components in green tea extract may be dissolved in oil containing compositions by using green tea extract on a carrier.

In one preferred embodiment said carrier is a polysaccharide, even more preferably said carrier is maltodextrin. In another embodiment, said carrier is an inorganic salt such as sodium chloride.

It is preferred that the composition of the present invention is subjected to some kind of treatment, e.g. filtration, in order to separate said carrier or at least the undissolved fraction of the carrier, from the composition. Accordingly, it is preferred that the composition of the present invention is free of carrier, at least free of any undissolved carrier.

The amount of said carrier is preferably in the range 50-95%, more preferably in the range 60-90%, even more preferably in the range 70-90%, most preferably about 80% by weight of the extract. Such an extract is commercially available under the trade name GUARDIAN™ Green Tea Extract 20M, from Danisco. An extract of this type can be used to prepare compositions according to the present invention in an amount of from 0.001 to 5% by weight of the composition, preferably in the range 0.002 to 1%, more preferably in the range 0.1 to 0.8% such as in the range 0.44 to 0.64%.

In one embodiment, at least additional 0.1 mg potassium hydroxide compared to the composition without green tea extract is required to neutralise 1 gram of the composition, more preferably at least additional 0.15 mg potassium hydroxide compared to the composition without green tea extract is required to neutralise 1 gram of the composition, even more preferably at least additional 0.2 mg potassium hydroxide compared to the composition without green tea extract is required to neutralise 1 gram of the composition and most preferably at least additional 0.25 mg potassium hydroxide compared to the composition without green tea extract is required to neutralise 1 gram of the composition.

In one embodiment, the composition according to the present invention comprises at least 0.05 mg catechins per gram composition (such as e.g. at least 0.06, 0.07, 0.08, 0.09, 0.1, 0.15, 0.2, 0.25, 0.3, 0.35, 0.4 or 0.45 mg catechins per gram composition), more preferably at least 0.5 mg catechins per gram composition (such as e.g. at least 0.55, 0.6, 0.65, 0.7, 0.75, 0.8, 0.85, 0.9 or 0.95 mg catechins per gram composition), even more preferably at least 1 mg catechins per gram composition (such as e.g. at least 1.05, 1.1, 1.15, 1.2, 1.25, 1.3, 1.35, 1.4 or 1.45 mg catechins per gram composition), most preferably at least 1.5 mg catechins per gram composition (such as e.g. at least 1.6, 1.8, 2, 2.2, 2.4, 2.6, 2.8, 3 or 4 mg catechins per gram composition). Said mg catechins per gram composition being analysed and calculated as set forth in example 7.

Further, the inventors of the present invention surprisingly discovered that if the green tea extract, or an active fraction thereof, is subjected to an extraction step, e.g. by using a lower alcohol (C₁₋₆ alcohol) such as e.g. ethanol; a filtration step; and that component A is added either to the filtered green tea extract or to the edible oil, the oil appeared totally clear with no visible particles and showed a significantly increased stability (example 9).

Thus, a fourth aspect of the present invention relates to a method for manufacturing the composition according to the present invention, the method comprising the following steps:
a) green tea extract or an active fraction thereof is mixed with a lower alcohol (C₁₋₆ alcohol), such as ethanol (e.g. abs. ethanol);
b) the product obtained in step a) is subjected to filtration, e.g. filtered on a nutch;
c) optionally, the filtrate obtained in step b) is mixed with ascorbyl palmitate;
d) component A, as defined in the third aspect of the present invention, or partial glycerides (monoglycerides and/or diglycerides, preferably monoglycerides), defined in the first aspect of the present invention, is added to the product obtained in step b) or step c); or optionally directly to the edible oil defined in step g);
e) optionally, at least one tocopherol or tocopherol derivative and/or rosemary extract or an active fraction thereof is added to the product obtained in step a), b), c) or d); and
f) the lower alcohol is removed;
g) the product obtained in step f) is mixed with an edible oil.

In one preferred embodiment, the filter cake obtained in step b) is washed with a lower alcohol (C₁₋₆ alcohol), such as ethanol (e.g. abs. ethanol), and the washing solution, combined with the filtrate obtained in step b).

In one preferred embodiment, ascorbyl palmitate is added to the filtrate obtained in step b) and the resulting solution shaken until all ascorbyl palmitate is dissolved.

Preferably, component A is added to the product obtained in step b) or step c) and the resulting mixture shaken vigorously to disperse component A.

The lower alcohol is preferably removed, e.g. at a in vacuo at a suitable temperature, or by any other method suitable to remove said lower alcohol.

In one preferred embodiment, the product obtained in step e) is mixed with rosemary extract (Rosemary Extract Liquide Refined, Vitablend) and tocopherol (Tocoblend L70 IP, Vitablend). The resulting mixture is then preferably homogenized, e.g. in vacuo at 60° C. on the rotary evaporator, until a viscous, transparent, deep red liquid is obtained.

As an alternative to the above disclosed method, the inventors of the present invention surprisingly also discovered that if a mixture of green tea extract and an edible oil is filtered using a filter having a pore size in the range 15-40 μm, the oil appeared totally clear with no visible particles and showed a significantly increased stability (example 1 and example 2).

Thus, a second aspect of the present invention relates to a method for manufacturing the composition according to the present invention, the method comprising the following steps:
a) optionally, green tea extract or an active fraction thereof is mixed with ascorbyl palmitate;
b) an edible oil is mixed with partial glycerides so that the final composition contains more than 2% partial glycerides (monoglycerides and/or diglycerides, preferably monoglycerides) by weight of the final composition; or an edible oil is mixed with component A, as defined in the third aspect of the present invention;
c) green tea extract or an active fraction thereof or the product obtained in step a) is mixed with the product obtained in step b);
d) the product obtained in step c) is subjected to filtration;
e) optionally, at least one tocopherol or tocopherol derivative and/or rosemary extract or an active fraction thereof is added to the product obtained in step a), b), c) and/or d).

Now being able to dissolve extracts from green tea in an edible oil, the inventors of the present invention have surprisingly found a synergistic antioxidative effect from combining a mixture of tocopherols, rosemary extract and ascorbyl palmitate with green tea extract.

Thus, it is preferred that the composition according to the present invention further comprises at least one tocopherol and/or tocopherol derivative; and/or rosemary extract or an active fraction thereof; and/or ascorbic acid and/or an ascorbic acid derivative.

By the expression “tocopherol” there is meant not only alpha-tocopherol but also beta-, gamma- or delta-tocopherol as well as any mixture thereof. 6-hydroxy-2,5,7,8-tetramethylchroman-2-carboxylic acid is also meant to be included in said group of tocopherol compounds.

The amount of tocopherol used in the composition will vary depending upon the potency of the chosen substance or mixture of substances, but will generally be in the range of from about 0.01-20% by weight of the composition, preferably in the range 0.01-5%, more preferably in the range 0.01-1%, even more preferably in the range 0.01-0.1%, most preferably about 0.1%. In one embodiment, there is more than one tocopherol in the mixture; particularly preferred is a mixture containing α-tocopherol, β-tocopherol, γ-tocopherol and δ-tocopherol. One example of such a mixture is Tocoblend L 70 IP from Vitablend. In another embodiment, γ-tocopherol is the only tocopherol present in the composition.

By the expression “rosemary extract or an active fraction thereof” there is meant any extracts obtainable from rosemary which have antioxidant activity. Antioxidant activity might be measured by a number of methods such as the Oxipres method disclosed in example 2.

It is preferred that said rosemary extract is an oil-soluble extract. Such rosemary extracts are commercially available from a variety of manufacturers. The preferred antioxidant fraction of the extract are primarily in the dehydroabeitic acid class of diterpenes. Among the specifically identified active ingredients of the extract are carnosol, carnosic acid and rosmanol. Thus, in one embodiment said extract, or an active fraction thereof, comprises carnosol, carnosic acid or rosmanol or any mixture thereof. However, there are other unidentified components of the extract which also possess antioxidant activity, and these may also be used in the composition.

One of the preferred rosemary extract is one which contains from about 1-5 (w/w) carnosic acid, from about 2-7% (w/w) carnosol, and from about 0.11-0.5% (w/w) rosmanol; such an extract is commercially available under the tradename StabexE; from SKW Chemicals. An extract of this type can be used in an amount of from about 0.0001 to about 1%, preferably about 0.1-0.5% by weight of the composition. Another preferred rosemary extract is an extract which is commercially available under the tradename Oleoresin Rosemary Herbalox® Brand, from Kalsec. Analysis of one batch of this extract showed it to contain approx. 4.1% carnosic acid, 0.61% carnosol and 0.034% rosmarinic acid. An extract of this type can be used in an amount of from about 0.0001 to about 1%, preferably about 0.1-0.5%, more preferably about 0.2-0.4%, e.g about 0.3% by weight of the composition. Alternatively one or more of the compounds carnosic acid, carnosol and/or rosmarinic acid may be added, preferably in a combined concentration from about 0.00005 to about 0.05%, preferably about 0.005-0.03%, more preferably about 0.01-0.02 by weight of the composition. Carnosic acid is the preferred compound to be added.

By the expression “ascorbic acid derivative” there is meant any derivative of ascorbic acid such as e.g. ascorbyl palmitate and ascorbyl stearate. Preferably said ascorbic acid and/or ascorbic acid derivative is ascorbyl palmitate. One example of a commercially available ascorbyl palmitate product is Grindox ascorbyl palmitate from Danisco. Preferably, said composition comprises ascorbic acid and/or an ascorbic acid derivative in the range of 0.02-0.12% by weight of the composition, preferably in the range 0.04-0.12% by weight of the composition, more preferably in the range 0.06-0.10%, even more preferably in the range 0.07-0.08%.

Further, it has previously been disclosed that ascorbyl palmitate is very effective in slowing down oxidation of lipids in marine oils. However, in order to dissolve this compound in oil formulations it is common to add lecithin, a compound which producers and distributors prefer to avoid since it has to be declared as a potential allergen.

Surprisingly, it has now been found that if said ascorbyl palmitate, preferably ground or milled ascorbyl palmitate, is premixed with a small amount of oil, preferably an oxidative stable oil (e.g. olive oil), the product is easily dissolvable in a marine oil.

By the expression “small amount of oil” there is meant that the volume of said oil is less than 1% (w/w) of the edible oil according to the present invention, preferably less than 0.1% (w/w), e.g. less than 0.01% (w/w).

Preferably, said ascorbic acid and/or a derivative thereof, the at least one tocopherol and/or tocopherol derivative, rosemary extract or an active fraction thereof is mixed with said green tea extract before filtering.

Further, it is preferred that both the ascorbic acid and/or a derivative thereof, the at least one tocopherol and/or tocopherol derivative, the rosemary extract or an active fraction thereof are dissolved in a small amount of oil. Said oil is preferably an oxidative stabile oil, such as a vegetable oil, e.g. olive oil such as food grade olive oil. To facilitate this process the oily mixture may be stirred, shaken and/or treated in an ultrasonic bath at temperature in the range 10-180° C. (such as 160° C.), e.g. in the range 20-120° C. (such as 20° C.), 50-100° C. or 80-90° C. (such as 80° C.).

Said ascorbic acid and/or a derivative thereof is preferably milled or ground before it is mixed with said small amount of an oil. Further, it is preferred that the milling or grounding is as gentle as possible, e.g. by a teflon coated magnet stirrer or a similar device suitable for large scale production.

Furthermore, it is preferred that said green tea extract is milled or ground.

In one preferred embodiment according to the present invention, the mixture that is to be filtered is stirred, shaken and/or treated in an ultrasonic bath at temperature in the range 10-180° C. (such as 160° C.), e.g. in the range 20-120° C. (such as 20° C.), 50-100° C. or 80-90° C. (such as 80° C.) before said mixture is filtered through a filter having a pore size in the range 0.1-100 preferably in the range 5-80 μm, more preferably in the range 10-50 μm and most preferably in the range 10-40 μm, such as 15-40 μm.

Thus, a further aspect of the present invention relates to a composition obtainable by the above mentioned methods.

There are a number of commercial antioxidant formulations suitable for improving oxidative stability of a marine oil, one of them being Tocoblend ATR. The latter, produced by the company Vitablend, is a commercial liquid antioxidant formulation containing the following ingredients: ascorbyl palmitate, natural mixed tocopherols, natural rosemary extract, polysorbate 80 and monopropylene glycol.

In order to compare Tocoblend ATR with the composition of the present invention, four formulation were made with the same batch of an omega-3-acid triglyceride concentrate (EPAX 6000TG, batch no. 2080630) complying with the European Pharmacopoeia monograph 1352, Omega-3-acid triglycerides (example 5). While the preparation based on the composition according to the present invention (preparation B) appeared as a suspension of finely divided particles before filtration, the preparation based on Tocoblend ATR (preparation D) formed sticky lumps of solids which did not disperse in the oil upon heating.

It is assumed that polysorbate 80 and/or monopropylene glycol in some manner interfere with the surface of the maltodextrin particles present in the green tea extract (GUARDIAN™ Green Tea Extract 20M, Danisco), resulting in some form of agglomeration which reduces the solubilisation of the green tea extract. Surprisingly, in Preparation B (examples 5) no such agglomeration occur, giving a product that is far better stabilised against oxidation.

In order to investigate this further, several commercially available emulsifiers and/or antioxidant formulations containing such emulsifiers were tested as additives in the preparation of compositions according to the present invention (example 6).

Surprisingly, these experiments indicate that the present invention has best effect when avoiding the common food additives like lecithin and polysorbate 80/monopropylene glycol. Contrary to expectations, a formulation with polysorbate 80/propylene glycol reduced the solubilisation of green tea extract. Other emulsifying agents, including the commonly used lechitin, increased problems with precipitation during storage of oil samples. On the other hand, the citric acid emulsifier gave excellent results in combination with a formulation according to the present invention (example 8 and 10).

Accordingly, it is preferred that the composition according to the present invention is essentially free of lechitin and/or polysorbate 80 and/or monopropylene glycol, more preferably the composition is totally free of lechitin and/or polysorbate 80 and/or monopropylene glycol. It is also preferred that said composition does not contain any synthetic emulsifiers.

Thus, in one preferred embodiment, the composition of the present invention further comprises citric acid esters of mono- and diglycerides. The skilled person will realise that other food additives/emulsifiers, such as e.g. tartaric acid esters of mono- and diglycerides and acetyltartaric acid esters of mono- and diglycerides, also may have similar effects as the citric acid based emulsifier.

A fifth aspect of the present invention relates to a nutritional, food or pharmaceutical composition comprising the composition according to the first or third aspect of the present invention or the composition obtained by the method according to the second or fourth aspect of the present invention.

The invention will now be described further with reference to the following non-limiting examples.

### EXAMPLES

### Example 1

### Preparation of Transparent Oil Samples Having Improved Oxidative Stability

### Preparation 1 (Ascorbyl Palmitate, Tocopherols and Rosemary Extract)

Ascorbyl palmitate (Grindox, Danisco) was milled to obtain a finely divided powder and mixed with tocopherol (Tocoblend L 70 IP, Vitablend), rosemary extract (Herbalox® Brand, type O, NS, Kalsec) and olive oil (Ybarra Virgin) by stirring at 50° C. for ½ hour. The weight ratio of the ingredients was ascorbyl palmitate (13.3%), tocopherol (19.1%), rosemary extract (53.2%) and olive oil (14.4%). 5.4 mg/g (mg per gram marine oil) of the combined ingredients were added to a marine oil (EPAX 6000 TG EPAX) by stirring at 80° C. for 20 minutes.

### Preparation 2 (Green Tea Extract)

5.4 mg/g (mg per gram marine oil) of a green tea extract (GUARDIAN™ Green Tea Extract 20M, Danisco) was stirred in a marine oil (EPAX 6000TG, EPAX) under heating at 80° C., and filtered while hot through a filter having a pore size in the range 15-40 μm.

### Preparation 3 (Cold Filtration)

5.4 mg/g of the combined ingredients as described in Preparation 1 and 5.4 mg/g of green tea extract were mixed with the same marine oil by stirring at 80° C. for 20 minutes. The preparation was cooled to 20° C. and filtered through a filter having a pore size in the range 15-40 μm.

### Preparation 4 (Hot Filtration)

5.4 mg/g of the combined ingredients as described in Preparation 1 and 5.4 mg/g of green tea extract were mixed with the same marine oil by stirring at 80° C. for 20 minutes. The preparation was filtered at 80° C. through a filter having a pore size in the range 15-40 μm.

### Example 2

### Oil Samples Having Improved Oxidative Stability (Oxipres Method)

The stability of the oils prepared in example 1 were tested by logging the pressure as a function of residence time at 50° C. and an oxygen pressure of about 3.5 bars. (Oxipres apparatus produced by Microlab, Aarhus, Denmark)

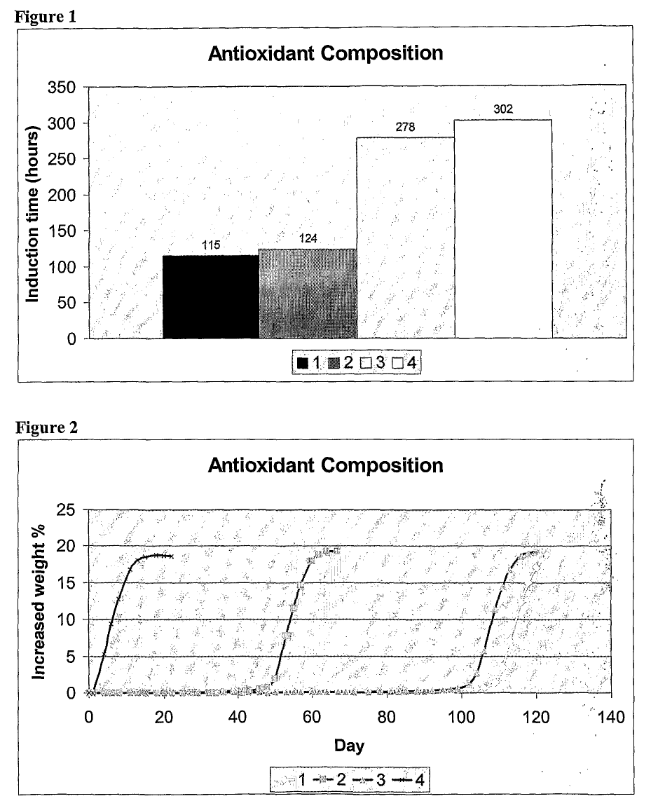

All preparations showed significant improved stability compared to the pure oil without any antioxidants. The induction time for Preparation 1 and Preparation 2 was 115 and 124 hours, respectively (FIG. 1). Preparation 3 (278 hours) showed less stability than Preparation 4 (302 hours) (FIG. 1).

Thus, the inventors of the present invention have found a synergistic antioxidative effect from combining a mixture of tocopherols, rosemary extract and ascorbyl palmitate with is green tea extract.

### Example 3

### Green Tea Extract with or without a Carrier

In Preparation 2 (example 1), a commercially available green tea extract which contains maltodextrin was used. In order to check whether maltodextrin is of importance or not, an analysis similar to what was done in example 2 was conducted using a commercially available green tea extract that does not contain a carrier (Indena™ Green Tea Extract). For comparison, the preparations were adjusted to contain the same amount of active ingredients. Even though the composition containing GUARDIAN™ Green Tea Extract 20M was shown to be more stable than the composition containing Indena™ Green Tea Extract, both products where demonstrated to be significantly more stable than the product which has not been mixed with green tea extract (data not shown). Further, maltodextrin alone does not affect the stability of the marine oil (Epax 6000TG oil, EPAX).

### Example 4

### Oil Samples Having Improved Oxidative Stability (Weight Increase Experiments)

Preparations similar to those described in Example 1 were made by using another marine oil, EPAX 6000 EE.

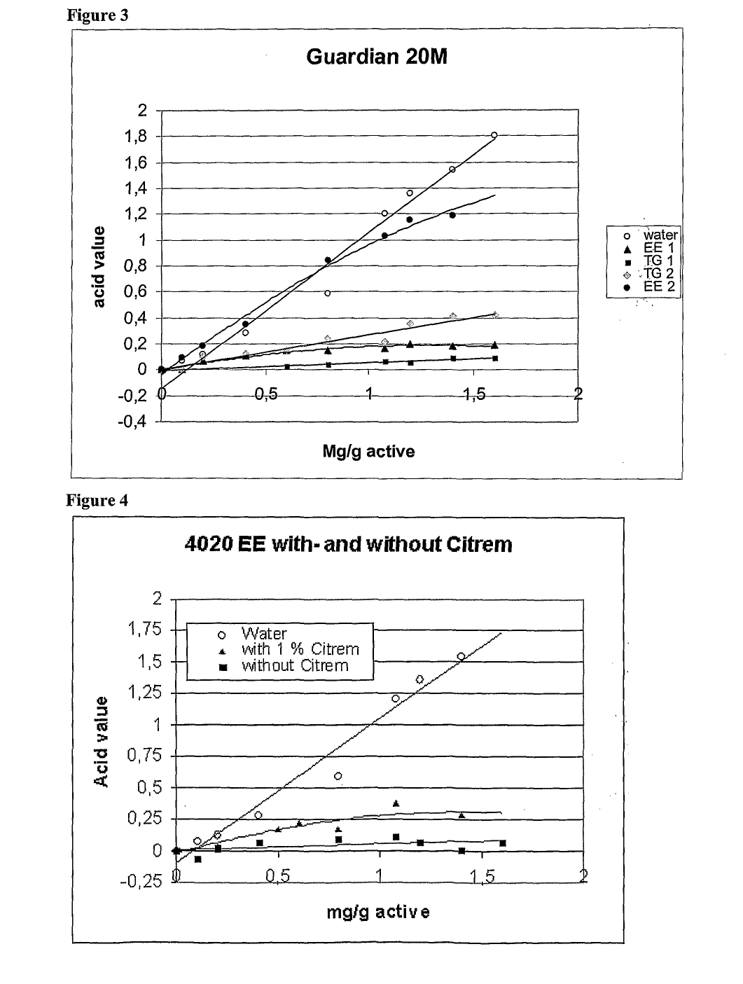

Samples (3.0 gram) are stored in Petri dishes (diameter 6 cm) and incubated in a cabinet at 30° C., ambient relative humidity. The increase in weight (%), as a result of oxidation, is plotted as a function of residence time (FIG. 2). The time span from start of the experiment to the point where the curve rises steeply, is defined as the induction time.

As can be seen (FIG. 2), the three preparations containing antioxidants are much more stable than the pure fish oil. However, the two preparations containing green tea extract is far more stable than the sample without green tea extract. In accordance with example 2, the inventors of the present invention have found a synergistic antioxidative effect from combining a mixture of tocopherols, rosemary extract and ascorbyl palmitate with green tea extract.

### Example 5

### Comparison with a Commercial Antioxidant Formulation (Oxipres Method)

### Preparation A (Ascorbyl Palmitate, Tocopherols and Rosemary Extract)

Ascorbyl palmitate (Grindox, Danisco) was milled to obtain a finely divided powder and mixed with tocopherol (Tocoblend L 70 IP, Vitablend), rosemary extract (Herbalox® Brand, type O, NS, Kalsec) and olive oil (Ybarra Virgin) by alternately magnetic stirring and immersing in a ultrasonic bath at 65° C. for 15 minutes. The weight ratio of the ingredients was ascorbyl palmitate (13.3%), tocopherol (19.1%), rosemary extract (53.2%) and olive oil (14.4%). 5.4 mg/g (mg per gram marine oil) of the combined ingredients were added to a marine oil (EPAX 6000 TG EPAX) by stirring at 80° C. for 20 minutes.

### Preparation B (Ascorbyl Palmitate, Tocopherols, Rosemary Extracts and Green Tea Extract)

Preparation B was prepared identical to Preparation 4 in Example 1.

### Preparation C (Tocoblend ATR¹)

5.4 mg/g (mg per gram marine oil) of Tocoblend ATR¹ was added to a marine oil (EPAX 6000 TG EPAX).

### Preparation D (Tocoblend ATR¹ and Green Tea Extract)

Preparation D was identical to preparation C, except that green tea extract was added in the same manner and in the same concentration as in Preparation B.

### ¹Tocoblend ATR

Tocoblend ATR, produced by the company Vitablend, is a commercial liquid antioxidant formulation containing the following ingredients: ascorbyl palmitate, natural mixed tocopherols, natural rosemary extract, polysorbate 80 and monopropylene glycol.

The stability of the oil preparations (A-D) were tested by the Oxipres method under is exactly the same conditions as described in example 2.

The induction time for Preparation A and Preparation B was 117 and 315 hours, respectively, while the induction time for Preparation C and Preparation D was 74 and 89 hours, respectively.

These results demonstrate that an antioxidant composition according to the present invention gives far better protection of omega-3-acid triglycerides than the commercial formulation Tocoblend ATR, also when the latter is combined with green tea dissolved from a carrier according to the present invention.

### Example 6

### Effect of Emulsifiers

### Lecithin

Addition of lecithin (l-α-lecithin, 3-sn-phosphatidylcholine from soy beans, Fluka) when making an antioxidant preparation as described in Preparation 1, Example 1 (relative weight of ascorbyl palmitate:lechitin=1:1) resulted in a product with a margarine like appearance. Although this formulation appeared less homogenous, and was more complicated to handle than the preparation 1 of Example 1, the formulation with lechitin was added to an omega-3 concentrate complying with the European Pharmacopoeia monograph 2063, Omega-3-acid ethyl esters 60.

Then green tea on maltodextrin was added under identical conditions as described in Preparation 4, Example 1. When stored in a refrigerator, precipitation of the oil was observed after only 6 days, making this preparation unsuitable for practical use. Similar precipitation, also after 6 days, was observed when the same oil, but with no addition of green tea, was stored in a refrigerator. When stored at room temperature, precipitate was formed after 6 days in the oil with green tea, and after 14 days in the oil without green tea.

Although lecithin is supposed to be a suitable additive in order to dissolve ascorbyl to palmitate, this Example surprisingly show that a preparation without lecithin is preferable for making compositions according to the present invention.

### Diacetyl Tartaric Acid Ester

An identical experiment was performed to that described for lecithin above, except that instead of lecithin a similar amount of the emulsifier Panodan® Visco-LO 2000, Danisco (diacetyl glycerides of tartaric acid made from sunflower oil) was utilised. The antioxidant formulation (including separate addition of green tea on a maltodextrin carrier according to the present invention) was added to an omega-3 concentrate complying with the European Pharmacopoeia monograph 2063, Omega-3-acid ethyl esters 60. Both when stored at room temperature and in refrigerator a precipitate was observed in the oil after only 6 days.

### Rosemary Extract Containing Diacetyl Tartaric Ester

An antioxidant formulation was prepared identical to Preparation 4 in Example 1, except that the rosemary extract was substituted with Rosemary extract liquid refined, containing the emulsifier diacatyl acid ester of mono and diglycerides (E472e) (Vitablend, article no. 51310). This antioxidant formulation was added to an omega-3 concentrate complying with the European Pharmacopoeia monograph 2063, Omega-3-acid ethyl esters 60. A precipitate occurred after storage in refrigerator for 7 days, indicating that even small amounts of the emulsifier diacetyl tartaric ester is negative for the applicability of antioxidant formulations according to the present invention.

### Acetic Acid Ester of Monoglycerides

Two identical experiments were performed to that described for diacetyl tartaric acid ester above, except that instead of diacetyl tartaric acid ester similar amounts of two commercial acetic acid esters of monoglycerides from hydrogenated palm based oil were used; the two acetic acid esters of monoglycerides had a degree of acetylation of 0.5 and 0.7 respectively. In both cases precipitate was observed after storage for 6 days, both at room temperature and in refrigerator.

### Citric Acid Ester of Mono- and Diglycerides

An identical experiment to that with acetic acid ester of mono- and diglycerides from hydrogenated palm oil were performed, except that instead of acetic acid ester the emulsifier Grinsted® Citrem 2-IN-1 cosher, Danisco was used. The product consists of a neutralised citric acid ester of mono-diglycerides. The formulation was added to two different batches of omega-3 concentrates complying with the European Pharmacopoeia monograph 2063, Omega-3-acid ethyl esters 60. When the experiments were ended, the oils had been stored at room temperatures at 11 weeks without formation of any visible precipitate.

### Example 7

### Effects of Partial Glycerides

The antioxidant effect of green tea has primarily been attributed to the polyphenol content of the tea leaves, commonly known as tea catechins. Based on the assumption that the active components of green tea extract to a large extent are present as compounds like phenols, the dissolved amount may be analysed by titration with potassium hydroxide.

### Preparation EE1

0, 0.5, 1, 2, 4, 5.4, 6, 7 and 8 mg/g (mg per gram marine oil) of a green tea extract (GUARDIAN™ Green Tea Extract 20M, Danisco) was stirred in a marine oil (5.6% triglycerides (TG); 3.0% diglycerides (DG); 3.6% monoglycerides (MG); 87.8% Ethyl esters/Free fatty acids (EE/FFA)) under heating at 80° C., and filtered while hot through a filter having a pore size in the range 15-40 μm.

### Preparation EE2

0, 0.5, 1, 2, 4, 5.4, 6, 7 and 8 mg/g (mg per gram marine oil) of a green tea extract (GUARDIAN™ Green TEA Extract 20M, Danisco) was stirred in a marine oil (0% triglycerides (TG); 0.5% diglycerides (DG),; 12.0% monoglycerides (MG); 87.5% ethylesters/Free fatty acids (EE/FFA)) under heating at 80° C., and filtered while hot through a filter having a pore size in the range 15-40 μm.

### Preparation TG1

3, 4, 5.4, 6, 7 and 8 mg/g (mg per gram marine oil) of a green tea extract (GUARDIAN™ Green Tea Extract 20M, Danisco) was stirred in a marine oil (93.6% triglycerides (TG); 4.9% diglycerides (DG); 0.4% monoglycerides (MG); 0.5% Ethyl esters/Free fatty acids (EE/FFA)) under heating at 80° C., and filtered while hot through a filter having a pore size in the range 15-40 μm.

### Preparation TG2

0, 0.5, 1, 2, 3, 4, 5.4, 6, 7 and 8 mg/g (mg per gram marine oil) of a green tea extract (GUARDIAN™ Green Tea Extract 20M, Danisco) was stirred in a marine oil (63.9% triglycerides (TG); 31.6% diglycerides (DG); 1.9% monoglycerides (MG); 2.5% Ethyl esters/Free fatty acids (EE/FFA)) under heating at 80° C., and filtered while hot through a filter having a pore size in the range 15-40 p.m.

### Preparation Water

0, 0.5, 1, 2, 4, 5.4, 6, 7 and 8 mg/g (mg per gram water) of a green tea extract (GUARDIAN™ Green Tea Extract 20M, Danisco) was stirred in water at room temperature.

### Preparation EE3

0, 0.5, 1, 2, 4, 5.4, 6, 7 and 8 mg/g (mg per gram marine oil) of a green tea extract (GUARDIAN™ Green Tea Extract 20M, Danisco) was stirred in a marine oil (0% triglycerides (TG); 0% diglycerides (DG); 0% monoglycerides (MG); 100% Ethyl esters/Free fatty acids (EE/FFA)) under heating at 80° C., and filtered while hot through a filter having a pore size in the range 15-40 p.m.

The contents of partial glycerides are analysed by size-exclusion chromatography similar to the method described in Ph. Eur. monographs 1352 and 2063.

Compared with the other preparations (EE1, TG1 and TG2), EE3 was shown to have the lowest amount of dissolved catechines (data not shown).

### Calculation of Dissolved Catechines

0, 0.5, 1, 2, 4, 5.4, 6, 7 and 8 mg/g (mg per gram water) of a green tea extract (GUARDIAN™ Green Tea Extract 20M, Danisco) was stirred in water at room temperature.

Each of the 9 samples is added potassium hydroxide until the composition is neutralised using phenolphthalein as indicator, cf. Ph. Eur. Monograph no. 2.5.1. Acid value.

The amount of added catechines (5.4 mg GUARDIAN™ Green Tea Extract 20M contains about 1.08 mg catechines) is plottet on the X-axis and the additional amount of potassium hydroxide compared to the composition without green tea extract necessary to neutralise the composition is plottet on the Y-axis as shown in FIG. 3.

Dissolution in water gives a straight line that can be utilised as a calibration curve for estimation of the dissolved amount in the omega-3 containing oils.

### Example 8

### Effects of Using Citric Acid Ester of Mono- and Diglycerides

The antioxidant effect of green tea has primarily been attributed to the polyphenol content of the tea leaves, commonly known as tea catechins. Based on the assumption that the active components of green tea extract to a large extent are present as compounds like phenols, the dissolved amount may be analysed by titration with potassium hydroxide.

0, 1, 2, 4 and 6 weight % Citrem was added to an ethyl ester (EPAX 4020 EE; no detectable amounts of partial glycerides; about 99% ethyl esters). The samples were split and half of the volumes retained as reference. To the other half were added antioxidants according to Example 1, Preparation 1 and 2, i.e. 5.4 mg/g of the mixture containing ascorbyl palmitate, tocopherols and rosemary extract, plus 5.4 mg/g GUARDIAN™ Green Tea Extract 20M, Danisco. All samples were heated to 80° C. for 20 minutes and filtered according to Preparation 4 (hot filtration). Acid numbers were determined according to Ph. Eur. Monograph 2.5.1.

TABLE 1

| | Acid |
|---|---|
| Composition | Value |
| Marine oil + ascorbyl palmitate + tocopherols + rosemary extract + | 1.1 |
| green tea extract | |
| Marine oil + ascorbyl palmitate + tocopherols + rosemary extract + | 1.6 |
| green tea extract + 1% Citrem by weight of the marine oil | |
| Marine oil + ascorbyl palmitate + tocopherols + rosemary extract + | 1.9 |
| green tea extract + 2% Citrem by weight of the marine oil | |
| Marine oil + ascorbyl palmitate + tocopherols + rosemary extract + | 2.3 |
| green tea extract + 4% Citrem by weight of the marine oil | |
| Marine oil + ascorbyl palmitate + tocopherols + rosemary extract + | 2.5 |
| green tea extract + 6% Citrem by weight of the marine oil | |
| Marine oil | 1.0 |
| Marine oil + 1% Citrem by weight of the marine oil | 1.1 |

As can bee seen from table 1, increasing concentration of Citrem results in an increased acid value which indicates that higher amounts of tea catechins have been dissolved in the marine oil.

### Example 9

### Improved Method for Preparation of Transparent Oil Samples

The preparation of transparent oil samples disclosed in example 1 involves filtration of the marine oil containing the specified antioxidants (“large” volumes). It has now surprisingly been found that filtration of the final composition is not required if the green tea extract is subjected to an extraction step, filtration step (small volume) and thereafter admixed with Citrem before it is mixed with the marine oil.

7.5 gram green tea extract (GUARDIAN™ Green Tea Extract 20M, Danisco) was stirred with 50 ml abs. ethanol and the resulting slurry filtered on a nutch. The filter cake was washed with a little abs. ethanol, and to the combined filtrates was added 2.1 gram ascorbyl palmitate (Grindox, Danisco). The filtrate was shaken until all ascorbyl palmitate was dissolved. The solution was transferred to a rotavapor flask and combined with 27 gram Citrem 2-in-1 (Danisco). The mixture was shaken vigorously to disperse the Citrem and ethanol removed in vacuo at 60° C. by spinning the flask on a rotary evaporator. To the residue was added 9.0 gram rosemary extract (Rosemary Extract Liquide Refined, Vitablend) and 3.0 gram tocopherol (Tocoblend L70 IP, Vitablend).

The resulting mixture was homogenized in vacuo at 60° C. on the rotary evaporator until a viscous, transparent, deep red liquid was obtained, 43.2 gram.

The oil appeared totally clear with no visible particles and showed a significantly increased stability (data not shown).

### Example 10

### Effects of Citric Acid Ester of Mono- and Diglycerides

The antioxidant effect of green tea has primarily been attributed to the polyphenol, content of the tea leaves, commonly known as tea catechins. Based on the assumption that the active components of green tea extract to a large extent are present as compounds like phenols, the dissolved amount may be analysed by titration with potassium hydroxide.

### Preparation 4020 EE

0, 0.5, 1, 2, 4, 6, 8 and 9 mg/g (mg per gram marine oil) of a green tea extract (GUARDIAN™ Green Tea Extract 20M, Danisco) with or without 1% citrem by weight of the marine oil was stirred in a marine oil (EPAX 4020EE; no detectable amounts of partial glycerides; about 99% ethyl esters) under heating at 80° C., and filtered while hot through a filter having a pore size in the range 15-40 μm.

### Preparation EPAX 6000 TG/N

0, 0.5, 1, 2, 4, 6, 8 and 9 mg/g (mg per gram marine oil) of a green tea extract (GUARDIAN™ Green Tea Extract 20M, Danisco) with or without 1% citrem by weight of the marine oil was stirred in a marine oil (EPAX 6000 TG/N; about 90% triglycerides and about 9% diglycerides) under heating at 80° C., and filtered while hot through a filter having a pore size in the range 15-40 μm.

As shown in FIGS. 4 and 5, the preparations containing 1% citrem by weight of the marine oil have higher amounts of dissolved cathechines compared with preparations without citrem. This effect has been demonstrated both in triglyceride oils (FIG. 5) and in ethyl ester oil (FIG. 4).

### Calculation of Dissolved Catechines

0, 0.5, 1, 2, 4, 6, 8 and 9 mg/g (mg per gram water) of a green tea extract (GUARDIAN™ Green Tea Extract 20M, Danisco) was stirred in water at room temperature.

Each of the 8 samples is added potassium hydroxide until the composition is neutralised using phenolphthalein as indicator, cf. Ph. Eur. Monograph no. 2.5.1. Acid value.

The amount of added catechines (5.4 mg GUARDIAN™ Green Tea Extract 20M contains about 1.08 mg catechines) is plottet on the X-axis and the amount of potassium hydroxide necessary to neutralise the composition* is plottet on the Y-axis as shown in FIGS. 4 and 5. * The amount of potassium hydroxide necessary to neutralise the composition is calculated as follows:
a) composition without Citrem: the amount of potassium hydroxide necessary to neutralise the composition−the amount of potassium hydroxide necessary to neutralise the marine oil;
b) composition with Citrem: the amount of potassium hydroxide necessary to neutralise the composition−the amount of potassium hydroxide necessary to neutralise the marine oil+Citrem.

Dissolution in water gives a straight line that can be utilised as a calibration curve for estimation of the dissolved amount in the omega-3 containing oils.

### Example 11

### Effects of Monoglycerides

The antioxidant effect of green tea has primarily been attributed to the polyphenol content of the tea leaves, commonly known as tea catechins. Based on the assumption that the active components of green tea extract to a large extent are present as compounds like phenols, the dissolved amount may be analysed by titration with potassium hydroxide.

### Preparation

0, 0.1, 0.6, 0.8, 1 and 1.4 mg/g (mg per gram EPAX 4020EE) of a green tea extract (GUARDIAN™ Green Tea Extract 20M, Danisco) was stirred in EPAX 4020EE (0% triglycerides (TG); 0 diglycerides (DG); 0% monoglycerides (MG); 100% Ethyl esters/Free fatty acids (EE/FFA)) under heating at 80° C., and filtered while hot through a filter having a pore size in the range 15-40 μm. One set of samples was added 2.1,% monoglycerides (said monoglycerides being prepared from EPAX 4020EE) by weight of the oily composition, and the other set of samples was not added any monoglycerides.

The amount of added catechines (5.4 mg GUARDIAN™ Green Tea Extract 20M contains about 1.08 mg catechines) is plottet on the X-axis and the additional amount of potassium hydroxide compared to the composition without green tea extract necessary to neutralise the composition is plottet on the Y-axis as shown in FIG. 6.

As shown in FIG. 6, the composition that contained monoglycerides was shown to have significantly higher amounts of dissolved catechines as compared to control.

1. Composition comprising an edible oil and green tea extract or an active fraction of said green tea extract, said composition containing more than 2% partial glycerides by weight of the composition.

2. The composition according to claim 1, wherein said partial glycerides are monoglycerides.

3. The composition according to claim 1, wherein said partial glycerides are diglycerides.

4. The composition according to claim 1, wherein said partial glycerides are a mixture of mono- and diglycerides.

5. The composition according to claim 1, wherein said partial glycerides are mono- and/or diesters of unsaturated fatty acids, preferably glycerol mono- and/or diesters of polyunsaturated fatty acids and most preferably glycerol mono- and/or diesters of omega-3 fatty acids.

6. The composition according to claim 1, wherein the composition contains more than 2.5% partial glycerides by weight of the composition, preferably at least 3% partial glycerides by weight of the composition, more preferably at least 5% partial glycerides by weight of the composition, even more preferably at least 8% partial glycerides by weight of the composition and most preferably at least 10% partial glycerides by weight of the composition.

7. Composition comprising an edible oil, green tea extract or an active fraction of said green tea extract and component A, wherein component A is a compound of formula (I) or a salt thereof; or a mixture of different compounds each represented by formula (I) or a salt thereof, 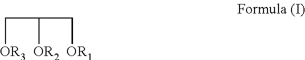
wherein
R₁ represents 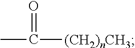
n is an integer in the range 3-30;
R₂ and R₃ are independently selected from the group consisting of 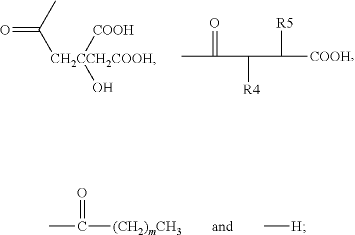
m is an integer in the range 3-30;
R₄ and R₅ are independently selected from 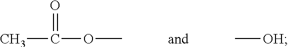
with the proviso that at least one of R₂ and R₃ is other than 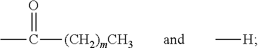
and that at least one of R₄ and R₅ is —OH.

8. The composition according to claim 7, wherein n is an integer in the range 12-28, more preferably in the range 14-24, even more preferably in the range 16-20 and most preferably n is 16.

9. The composition according to claim 7, wherein m is an integer in the range 12-28, more preferably in the range 14-24, even more preferably in the range 16-20 and most preferably n is 16.

10. The composition according to claim 7, wherein component A represents a compound of formula (II) or a salt thereof; a compound of formula (III) or a salt thereof; or any mixture thereof

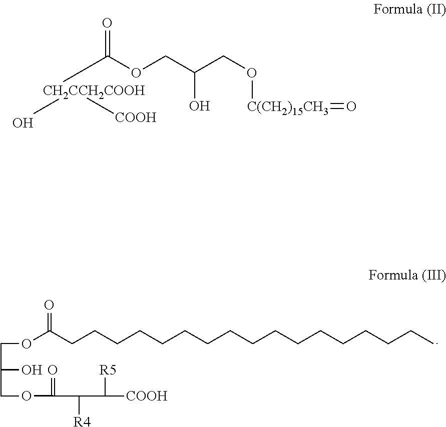

11. The composition according to claim 7, wherein the composition contains at least 0.3% component A by weight of the composition, preferably at least 0.35% component A by weight of the composition, more preferably at least 0.4% component A by weight of the composition, even more preferably at least 0.5% component A by weight of the composition and most preferably at least 0.55% component A by weight of the composition such as 0.6%, 0.65%, 0.7%, 0.75%, 0.8%, 0.85%, 0.9%, 0.95%, 1%, 2%, 3%, 4%, 5% or 6% by weight of the composition.

12. The composition according to claim 7, wherein said component A is essentially in neutralized form.

13. The composition according to any one of claims 1-12, wherein said edible oil is a marine oil, preferably fish oil.

14. The composition according to any one of claims 1-12, wherein said edible oil contains >70% ethyl esters by weight of the edible oil, said ethyl esters preferably being ethyl esters of omega-3 fatty acids.

15. The composition according to any one of claims 1-12, wherein said edible oil contains >60% omega-3 fatty acids by weight of the edible oil.

16. The composition according to any one of claims 1-12, wherein said green tea extract or an active fraction of said green tea extract further comprises a carrier, said carrier preferably being a polysaccharide or a mixture of polysaccharides, more preferably said carrier is maltodextrin.

17. The composition according to claim 16, wherein undissolved carrier is removed from the composition, e.g. by means of filtration.

18. A method for manufacturing the composition according to any one of claims 1-17, the method comprising the following steps:
a) optionally, green tea extract or an active fraction thereof is mixed with ascorbyl palmitate;
b) an edible oil is mixed with partial glycerides so that the final composition contains more than 2% partial glycerides by weight of the final composition; or an edible oil is mixed with component A, as defined in claim 7;
c) green tea extract or an active fraction thereof or the product obtained in step a) is mixed with the product obtained in step b);
d) the product obtained in step c) is subjected to filtration;
e) optionally, at least one tocopherol or tocopherol derivative and/or rosemary extract or an active fraction thereof is added to the product obtained in step a), b), c) and/or d).

19. A method for manufacturing the composition according to any one of claims 1-17, the method comprising the following steps:
a) green tea extract or an active fraction thereof is mixed with a lower alcohol, such as ethanol;
b) the product obtained in step a) is subjected to filtration;
c) optionally, the filtrate obtained in step b) is mixed with ascorbyl palmitate;
d) component A, as defined in claim 7, or partial glycerides, as defined in claim 1, is added to the product obtained in step b) or c);
e) optionally, at least one tocopherol or tocopherol derivative and/or rosemary extract or an active fraction thereof is added to the product obtained in step a), b), c) or d);
f) the lower alcohol is removed;
g) the product obtained in step f) is mixed with the edible oil.

20. A nutritional, food or pharmaceutical composition, comprising the composition according to any one of claims 1-17 or the composition obtainable by the method according to any one of claims 18-19.
